(19) United States
(12) Patent Application Publication
(10) Pub. No.: US 2008/0255455 A1
(43) Pub. Date: Oct. 16, 2008

(54) Probe holder for portable diagnostic ultrasound system

(75) Inventors: Alexander Sokulin, Kiryat Tivon (IL); Mark Dahan, Haifa (IL); Michal Binenbaum, Zichron Yaakov (IL); Moshe Sela, Haifa (IL)

Correspondence Address: DEAN D. SMALL;THE SMALL PATENT LAW GROUP LLP, 225 S. MERAMEC, STE. 725T, ST. LOUIS, MO, 63105, US

(73) Assignee: GENERAL ELECTRIC COMPANY

(21) Appl. No.: 11/786,331

(22) Filed: Apr. 11, 2007

(51) Int. Cl. A61B 8/00

(52) U.S. Cl. 600/459

(57) ABSTRACT

An ultrasound probe holder for use with a portable ultrasound system comprises a probe holder that is configured to have first and second interconnection portions. The first interconnection portion is configured to interconnect with a three-dimensional (3D) object. A second opening is formed in the second interconnection portion, and is configured to accept a scanning end of an ultrasound probe.

### BACKGROUND OF THE INVENTION

This invention relates generally to portable and handheld ultrasound systems, and more specifically, to supports for probes used with portable and handheld ultrasound systems.

Ultrasound probes have scanning and connector ends that are connected together with a long cable. Conventional and cart-based ultrasound systems often provide multiple ports that accept multiple connector ends of probes. When a probe connected to the system is not in use, the scanning end of the probe may be placed in a probe holder mounted on the console of the system to prevent falling and/or damage. Multiple probe holders may be provided for the different probes used during different exam types. Each probe holder may be a cup-shaped or C-shaped device that holds the scanning end of the probe in an upright position. An opening or slot at the bottom end of the device allows the cord of the probe to hang vertically.

Portable and handheld ultrasound scanning systems are becoming more popular and may be similar in size and shape to a laptop computer or even smaller, such as small enough to fit inside a user's pocket. In such systems, a single port may be provided to accept the ultrasound connector of a probe, but no provision has been made to hold the probe. Ultrasound probes are expensive and fragile, and may be difficult to protect when the user is not actively scanning the patient with the scanning end.

Attaching a traditional probe holder onto a portable or handheld ultrasound system may hamper the portability of the system by making the system much larger. Also, a traditional probe holder would limit where the portable system may be placed as the probe cord extends from the bottom end of the probe holder. Traditional probe holders are deep enough that the probe will not fall out from the top and thus will typically loosely accept the scanning end. This is acceptable when the holder is fixed to a cart-based console as tipping is not an issue. However, the portable ultrasound system may be tipped and/or held and carried at any angle such that probes placed in traditional probe holders may fall out, thereby resulting in potential damage to the probe.

Also, in some cases the user may wish to transport the portable system along with more than one ultrasound probe. In general, loosely carrying the probes is awkward due to the configuration of two pieces mounted at either end of the long cable. Additionally, the cables of the probes may become tangled and difficult to deal with when carrying multiple probes at a time.

Therefore, a need exists for securely handling ultrasound probes when using a portable ultrasound system.

### BRIEF DESCRIPTION OF THE INVENTION

In one embodiment, an ultrasound probe holder for use with a portable ultrasound system comprises a probe holder that is configured to have first and second interconnection portions. The first interconnection portion is configured to interconnect with a three-dimensional (3D) object. A second opening is formed in the second interconnection portion, and is configured to accept a scanning end of an ultrasound probe.

In another embodiment, a portable ultrasound system comprises a portable ultrasound system for acquiring ultrasonic data of a patient. An ultrasound probe is connectable to the ultrasound system for scanning the patient. The ultrasound probe has a connector end for interfacing with the system and a scanning end for scanning the patient. The connector end and the scanning end are interconnected with a cable. A probe holder holds at least the scanning end of the ultrasound probe when the scanning end is not being used to scan the patient.

In yet another embodiment, an ultrasound probe holder comprises a central region having a top and a bottom that are opposite to each other and first and second sides that are opposite to each other. Top and bottom portions extend from the central region in both first and second directions. The top and bottom portions form first and second openings in the first and second sides, respectively. The first and second openings are configured to releasably hold a connector end and a scanning end, respectively, of an ultrasound probe.

### BRIEF DESCRIPTION OF THE DRAWINGS

FIG. 1 illustrates a portable ultrasound system formed in accordance with an embodiment of the present invention.

FIG. 2 illustrates an example of a pocket-sized ultrasound system formed in accordance with an embodiment of the present invention.

FIG. 3 illustrates a block diagram of functionality that may be provided within the portable systems of FIGS. 1 and 2 in accordance with an embodiment of the present invention.

FIG. 4 illustrates a probe holder that may be used with the portable systems of FIGS. 1 and 2 in accordance with an embodiment of the present invention.

FIG. 5 illustrates the portable ultrasound system of FIG. 1 interfacing with a probe and probe holder in accordance with an embodiment of the present invention.

FIG. 6 illustrates a geometric view of a probe holder formed in accordance with an embodiment of the present invention.

FIG. 7 illustrates a cross-sectional view of the probe holder of FIG. 6 formed in accordance with an embodiment of the present invention.

FIG. 8 illustrates an alternative probe holder formed in accordance with an embodiment of the present invention for holding the scanning end of the probe and interconnecting with another object.

FIG. 9 illustrates another alternative probe holder formed in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

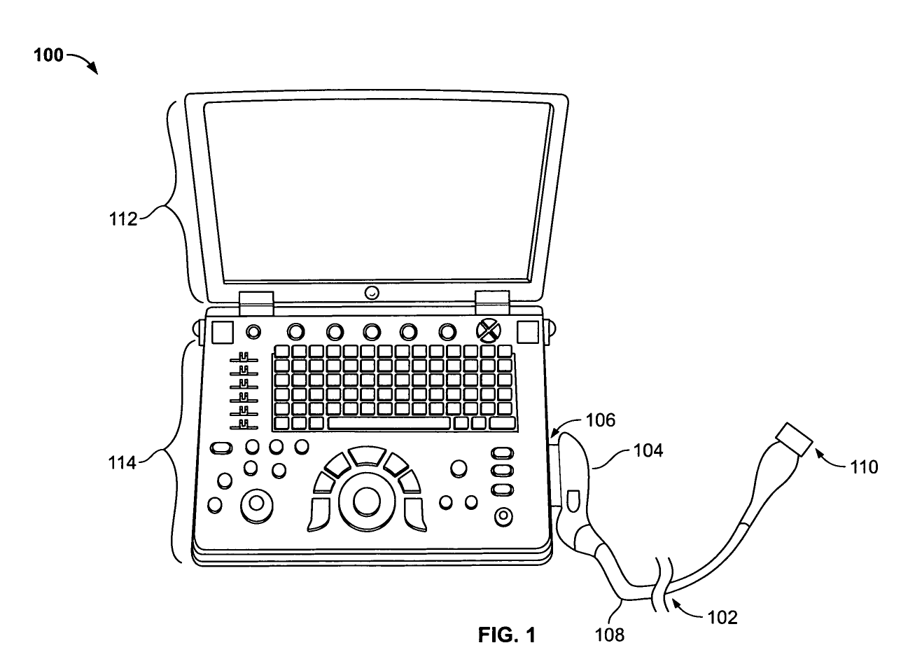

FIG. 1 illustrates a miniaturized ultrasound system 100. As used herein, “miniaturized” means that the ultrasound system is a handheld or hand-carried device or is configured to be carried in a person's hand, pocket, briefcase-sized case, or backpack. For example, the ultrasound system 100 may be a hand-carried device having a size of a typical laptop computer, for instance, having dimensions of approximately 2.5 inches in depth, approximately 14 inches in width, and approximately 12 inches in height. The ultrasound system 100 may weigh about ten pounds.

An ultrasound probe 102 has a connector end 104 that interfaces with the system 100 through an I/O port 106 on the system 100. The probe 102 has a cable 108 that connects the connector end 104 and scanning end 110 that is used to scan a patient. The system 100 also has a display 112 and a user interface 114.

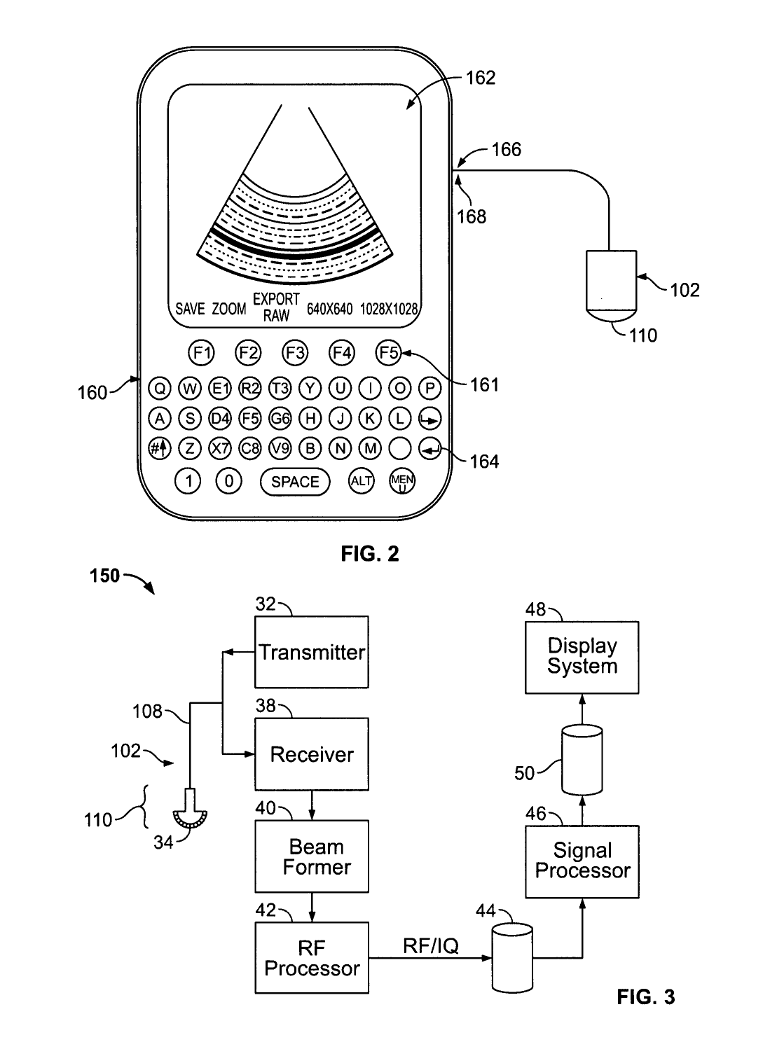

FIG. 2 shows an example of a pocket-sized ultrasound system 160. By way of example, the pocket-sized ultrasound system 160 may be approximately 2 inches wide, approximately 4 inches in length, and approximately 0.5 inches in depth and weigh less than 3 ounces. The pocket-sized ultrasound system 160 generally includes a display 162, a user interface 164 (e.g., keyboard) and an input/output (I/O) port 166 for connection to the probe 102. In one embodiment, the system 160 may be configured to operate with a single probe 102 having a connector end 168 of the probe 102 that may be hard-wired to the system 160 rather than interconnected through the port 166. It should be noted that the various embodiments may be implemented in connection with a miniaturized ultrasound system having different dimensions, weights, and power consumption. In some embodiments, the pocket-sized ultrasound system 160 may provide the same functionality as the system 100 of FIG. 1.

FIG. 3 illustrates a block diagram of functionality that may be provided within the systems 100 and 160 of FIGS. 1 and 2, respectively. It should be understood that the system architecture and functionality illustrated and discussed herein are exemplary and not limiting. For example, a function such as beamforming may be accomplished separate from or may be integrated partially or fully together with the probe 102. The ultrasound system 150 includes a transmitter 32 that drives transducer elements 34 within the scanning end 110 of the probe 102 to emit pulsed ultrasonic signals into a body. The transducer elements 34 include piezoelectric (or other) elements (not shown) that fire an ultrasound pulse. A variety of geometries for transmitting the ultrasound signals may be used. For example, the probe 102 may be a curved linear probe, a linear probe or a phased array probe. The ultrasonic signals are back-scattered from structures in the body, like blood cells or muscular tissue, to produce echoes which return to the transducer elements 34. The echoes are received by a receiver 38, and are passed through a beamformer 40 that performs beamforming and outputs an RF signal. The RF signal then passes through an RF processor 42. Alternatively, the RF processor 42 may include a complex demodulator (not shown) that demodulates the RF signal to form IQ data pairs representative of the echo signals. The I and Q values of the beams represent in-phase and quadrature components of a magnitude of echo signals reflected from a point P at a range R and an angle θ. The RF or IQ signal data may then be routed directly to a RF/IQ buffer 44 for temporary storage.

A signal processor 46 generally processes the acquired ultrasound information (i.e., RF signal data or IQ data pairs) and prepares frames of ultrasound information for display on a display system 48. The signal processor 46 is adapted to perform one or more processing operations (e.g., compounding) according to a plurality of selectable ultrasound modalities on the acquired ultrasound information. Acquired ultrasound information may be processed in real-time during a scanning session as the echo signals are received. Additionally or alternatively, the ultrasound information may be stored temporarily in RF/IQ buffer 44 during a scanning session and processed in less than real-time in a live or off-line operation.

The ultrasound system 150 may continuously acquire ultrasound information at a frame rate that exceeds fifty frames per second, which is the approximate perception rate of the human eye. The acquired ultrasound information is displayed on the display system 48 at a slower frame-rate. A memory 50 optionally is included for storing processed frames of acquired ultrasound information that are not scheduled to be displayed immediately. Preferably, the memory 50 is of sufficient capacity to store at least several seconds worth of frames of ultrasound information. The frames of ultrasound information are stored in a manner to facilitate retrieval thereof according to its order or time of acquisition. The memory 50 may comprise any known data storage medium.

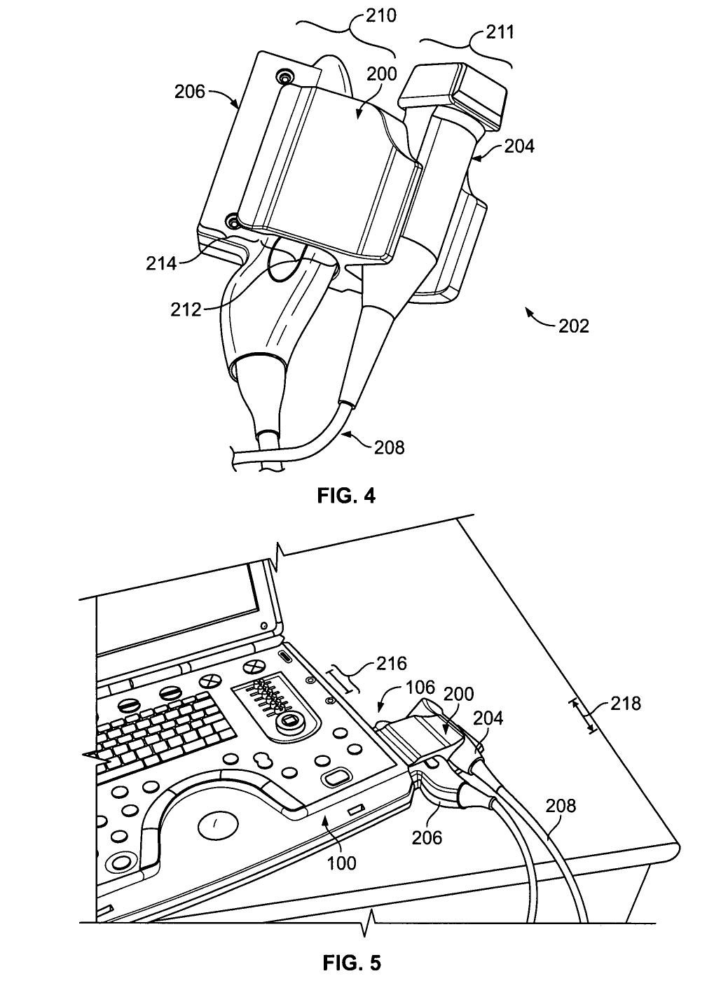

FIG. 4 illustrates a probe holder 200 that may be used with the portable systems 100 and 160 of FIGS. 1 and 2, respectively. In this embodiment, ultrasound probe 202 has a scanning end 204 for scanning a patient and a connector end 206 that connects, for example, to the I/O port 106 of the ultrasound system 100 of FIG. 1. The probe holder 200 has a first interconnection portion 210 that receives a portion of the connector end 206. The first interconnection portion 210 and connector end 206 may interconnect with removable engagement such as sliding together, snap fit, tension fit, and the like. Thus, a releasable attachment is provided.

Cable 208 interconnects the scanning end 204 and the connector end 206 of the probe 202, facilitating the transfer of signals. Although not fully shown, the cable 208 may be long (e.g., 3 meters in length). The probe holder 200 has a second interconnection portion 211 that accepts the scanning end 204 of the probe 202, such as with a tension fit. The scanning end 204 and the connector end 206 may thus be held together to form a single assembly, making the probe 202 easier to handle and/or transport. Additionally, the cable 208 may be prevented from becoming tangled with other probe cables.

Turning to FIG. 5, the user may engage (e.g., push into) the connector end 206 of the probe 202 with the I/O port 106 on the system 100 (or with the I/O port 166 of the system 160 of FIG. 2). This engagement may be provided, for example, by complementary connection members. Other mating connections may be used. An additional latching mechanism (not shown) may be provided to ensure that the connector end 206 and I/O port 106 are mated properly and securely with each other and will not become separated unintentionally. Previously, when not in use, the scanning end 204 of the probe 202 may be set on a table, desk, or patient bed, such as when the user is entering data into the system 100. The scanning end 204 may then be damaged by falling and/or having items set upon and/or fall upon the scanning end 204. By using the probe holder 200, the scanning end 204 is securely held when not in use.

Alternatively, the probe holder 200 may be configured to hold the scanning end 204 on one side (corresponding to the second interconnection portion 211) and attach to a different three-dimensional object on the other side (corresponding to the first interconnection portion 210). For example, the probe holder 200 may attach to the ultrasound system 100, such as along a system edge 216. Optionally, the probe holder 200 may attach to a desk or table top 218. In another embodiment, the probe holder 200 may attach to a case (not shown) used to carry the ultrasound probe and/or the system 100 or 160.

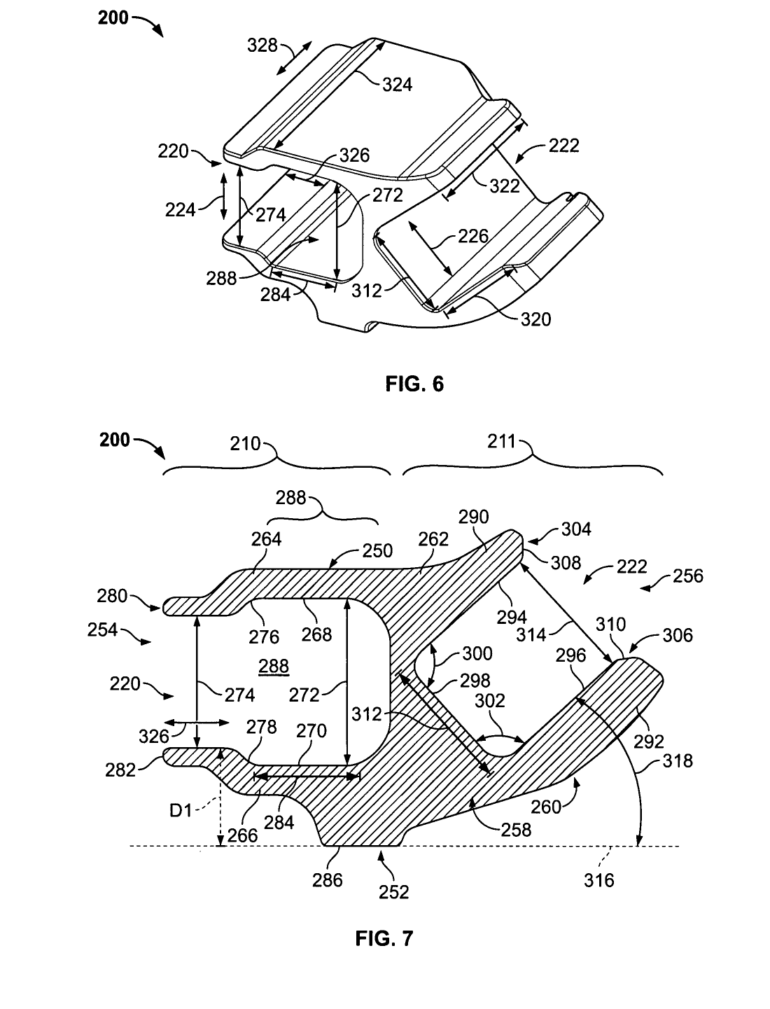

FIG. 6 illustrates the probe holder 200 of FIG. 4. The first interconnection portion 210 of the probe holder 200 has a first opening 220 for receiving the connector end 206 of the probe 202 and the second interconnection portion 211 has a second opening 222 for receiving the scanning end 204 of the probe 202. Alternatively, rather than attaching the probe holder 200 to the connector end 206 of the probe 202, the probe holder 200 may be configured to attach to an edge of the ultrasound system 100 or 160 and/or to an edge of a table top. In one embodiment, if a portable ultrasound system 160 is configured to operate with a single probe that is hard-wired thereto as discussed previously, a releasable connector end 206 may not be available and the probe holder 200 may be attached to an edge of the system 160.

The geometry and/or size of the first opening 220 may be based on the external geometry of the connector end 206 or on a system or table edge to be inserted into the first opening 220. The geometry of the second opening 222 may be based on the external geometry of the scanning end 204 of the probe 202 to be held by and/or secured by the second opening 222. For example, first and second probes may have first and second geometries, respectively, that define external dimensions of the connector and scanning ends. A first probe holder may be configured based on the first geometry and a second probe holder may be configured based on the second geometry. By way of example only, the first and second probes may have the same external geometry with respect to one of the connector and scanning ends while the other end is different.

The probe holder 200 may be formed from a single piece of material, such as by molding or other process (e.g., single unitary construction). Alternatively, the probe holder 200 may be an assembly of two or more pieces that may be held together with one or more fastener, such as screws, glue, adhesive and the like. Alternatively, the two or more pieces may be formed to be interlocking with one another. The material may be a plastic material or any other material that provides desired properties. The material may have the ability to flex or give slightly under force as the connector and scanning ends 206 and 204 are inserted into the first and second openings 220 and 222, respectively. The material returns to the original shape and geometric measurements when the force is removed. The first and second openings 220 and 222 also may have a coating or material that further maintains engagement of the probe holder 200 to the probe 202.

The external geometry of the connector end 206 as shown in FIG. 4 illustrates wider and narrower areas 212 and 214. In FIG. 6, inner height 272 and depth 284 dimensions of an inner portion 288 of the first opening 220 are based on the dimensions of the wider area 212 of the connector end 206. Outer height 274 and depth 326 of the first opening 220 are based on the narrow area 214 of the connector end 206. The first opening 220 also has a length 324 dimension based on the external geometry of the connector end 206.

The connector end 206 of the probe holder 200 may slide over the connector end 206 in the direction of arrow 328 such that the wider area 212 of the connector end 206 is held within the inner portion 288. The inner portion 288 may be configured to hold the wider area 212 with some tension and/or friction. Alternatively, the connector end 206 may be pushed into the first opening 220. In this example, the first opening 220 flexes outward slightly in the directions of arrow 224 and the wider area 212 is received and held in the inner portion 288 of the first opening 220. When the force along arrow 224 is removed, the material returns to its original position, securely holding the wider area 212 of the connector end 206 within the first opening 220. Similarly, when the user pulls the connector end 206 and the probe holder 200 apart, the second opening 222 flexes in the directions of the arrow 224 to allow the removal of the connector end 206.

The second opening 222 is formed having depth 320, length 322, and inner height 312 dimensions that are based on the scanning end 204 of the probe 202. Although not shown, the scanning end 204 may have an external geometry providing flat surfaces that interface with sides of the second opening 222 along the depth 320. When the user pushes the scanning end 204 into the second opening 222, the material flexes outward in the directions of arrow 226. The second opening 222 exerts a tension on the scanning end 204 to hold the scanning end 204 within the second opening 222.

A user may insert and remove the scanning end 204 of the probe 202 multiple times during a scan of the same patient. In an embodiment wherein the connector end 206 is pushed into the probe holder 200 as discussed previously, the probe holder 200 may be configured such that less force is needed to remove the scanning end 204 from the second opening 222 than is needed to remove the connector end 206 from the first opening 220 to ensure that the probe holder 200 remains connected to the connector end 206. The second opening 222 is also configured to exert enough force on the scanning end to retain the scanning end 204 regardless of the orientation of the probe holder 200.

FIG. 7 illustrates a cross-sectional view of the probe holder 200 that has, generally, a top 250, bottom 252, first side 254, second side 256, front side 258 and back side 260. The bottom 252 may have a flat portion 286 configured to rest on a table top. This may alleviate mechanical stress between the connector end 206 and I/O port 106 by holding the components in a desired relationship with respect to each other. For example, when the probe holder 200 is interconnected with the system 100 as illustrated in FIG. 5 and the system 100 is placed on a surface, the flat portion 286 is a distance D1 that is based on a distance to a bottom (not shown) of the system 100.

The first and second openings 220 and 222 are indicated in the first and second sides 254 and 256, respectively, both of which extend from a central region 262 of the probe holder 200. The first and second openings 220 and 222 are open to both the front and back sides 258 and 260.

Top and bottom portions 264 and 266 extend substantially parallel with respect to each other from the central region 262 to form the first opening 220. The top and bottom portions 264 and 266 are curved to form lips 276 and 278, respectively. Top and bottom inner surfaces 268 and 270 are separated by the inner height 272 proximate to the central region 262 (forming inner portion 288) and the outer height 274. The inner height 272 is larger than the outer height 274, and thus the top and bottom portions 264 and 266 form a C-clamp shape. The depth 284 and length 324 of the inner portion 288 are also illustrated. In one embodiment, first and second leading edges 280 and 282 of the top and bottom portions 264 and 266 may be rounded to facilitate the insertion of the connector end 206. The top and bottom portions 264 and 266 move outward with respect to each other to allow the wider area 212 of the connector end 206 having the dimensions of the inner height 272 to pass through. When the connector end 206 is fully inserted, the material of the top and bottom portions 264 and 266 retains the original shape and geometry to hold the connector end 206 within the first opening 220 (e.g., resilient operation).

Alternatively, the top and bottom portions 264 and 266 may not be curved, such as when the first opening 220 is configured to receive an edge of the system 100 or an edge of a table top that may be substantially rectangular in shape. The outer height 274 may be slightly less than the inner height 272, and the first opening 220 may be configured to exert a force on the inserted object as discussed previously with the second opening 222.

Top and bottom portions 290 and 292 extend from the central region 262 of the probe holder 200 to form the second opening 222. The second opening 222 is substantially U-shaped. The top and bottom portions 290 and 292 have top and bottom inner surfaces 294 and 296, respectively. First and second leading edges 304 and 306 of the top and bottom portions 290 and 292 have first and second beveled edges 308 and 310, respectively, proximate to the second opening 222 to facilitate the insertion of the scanning end 204.

Back wall 298 extends to each of the top and bottom inner surfaces 294 and 296 and forms first and second angles 300 and 302. One of the first and second angles 300 and 302 may be less than 90 degrees while the other angle is approximately 90 degrees. In one embodiment, the first angle 300 is about 87 degrees and the second angle 302 is about 90 degrees. Therefore, the top and bottom inner surfaces 294 and 296 are not parallel with respect to each other and the top inner surface 294 extends slightly into the second opening 222. Also, inner height 312 of the second opening 222 is slightly greater than outer height 314. It should be understood that the first and second angles 300 and 302 are not limited to those discussed herein and may be larger or smaller. Also, the first and second angles 300 and 302 may be the same or different.

The second opening 222 is formed to open upwards towards the top 250 and the second side 256 while the first opening 220 opens towards the first side 254. For reference, bottom plane 316 is illustrated extending parallel to the surface of the bottom 252 of the probe holder 200. The bottom inner surface 296 may form an angle 318 with the bottom plane 316 of approximately 45 degrees. The angle 318 may be more or less than 45 degrees, and is not limited to the illustrated embodiment. By tilting the second opening 222 in an upwards direction, the user may more easily insert and remove the scanning end 204 of the probe 202 while scanning a patient. Also, when held in the probe holder 200, the scanning end 204 is prevented from contacting a surface that the system 100 is on that may necessitate cleaning the scanning end 204 before returning to scanning the patient.

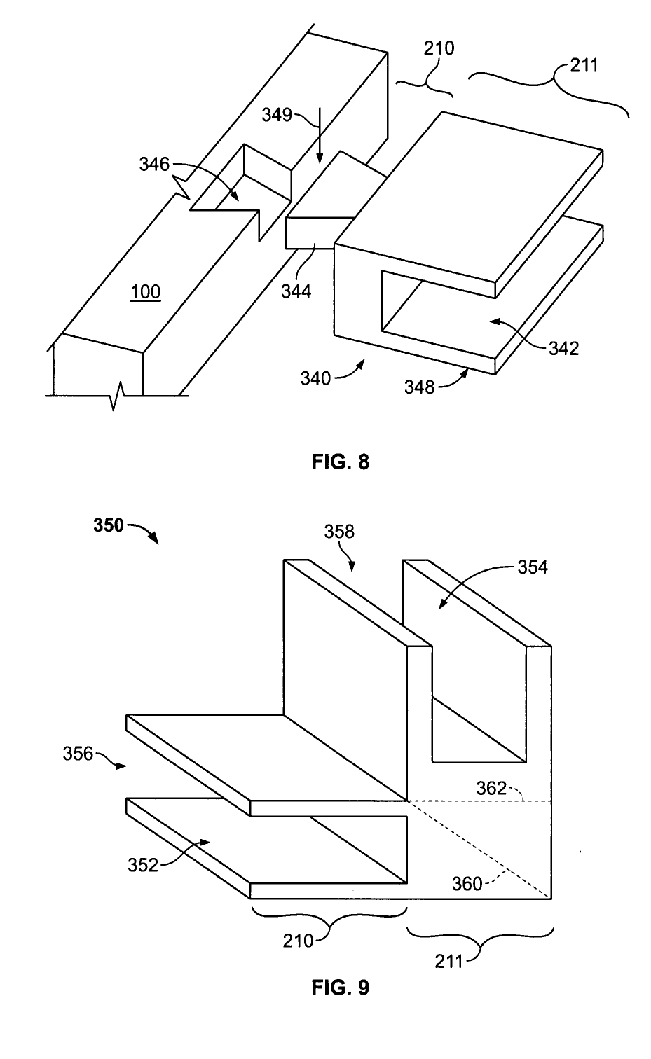

FIG. 8 illustrates an alternative probe holder 340 having an opening 342 for receiving the scanning end 204 of the probe 202 and an interconnecting portion 344 that is received by a port or opening. Therefore, the second interconnection portion 211 of the probe holder 340 accepts the scanning end 204 of the probe 202 and the first interconnection portion 210 interconnects with another 3D object. For example, opening 346 may be provided on the ultrasound system 100 of FIG. 1, an ultrasound system or probe carrying case, and the like. The interconnecting portion 344 is inserted into and securely held by the opening 346. For example, the interconnecting portion 344 may be inserted into the opening 246 in the direction of arrow 349. The probe holder 340 may be configured such that one side 348 rests on a table top or other surface when the interconnecting portion 344 and opening 346 are mated together.

FIG. 9 illustrates an alternative probe holder 350. The first interconnection portion 210 of the probe holder 350 has a first opening 352 configured to accept the connector end 206 of the probe 202, an edge of the ultrasound system 100 or 160, and/or an edge of a table top. The second interconnection portion 211 has a second opening 354 that is configured to accept the scanning end 204 of the probe 202. The first and second openings 352 and 354 are generally on first and second sides 356 and 358 of the probe holder 350. In this example, the second opening 354 is formed in a substantially upward direction, however, the second opening 354 is not limited to the displayed orientation.

The probe holder 350 may be formed of two or more pieces attached to one another. In general, a joint may be formed, such as along either line 360 or 362, to join multiple separate pieces together. As discussed previously, the multiple pieces may be joined with screws, adhesive, a mechanical interlocking member, or be formed to snap or interlock together. Alternatively, the probe holder 350 may have a unitary construction, such as to be formed of a single piece of material. Other constructions and orientations may be used and are not limited to those discussed herein.

A technical effect of at least one embodiment is holding an ultrasound scanning probe securely when using a portable ultrasound scanning device. The probe holder provides a safe and secure place for the user to place the scanning end of the probe when not in use, while still allowing the user to easily remove the scanning end from the probe connector in a manner that facilitates quickly examining a patient. Also, when the probe holder is attached to a probe connector that is interconnected with the portable ultrasound system, the probe holder holds the scanning end securely so that a user may more easily move from one room to another. Additionally, the probe holder may be used to attach the connector and scanning ends of a probe to one another to form a single unit to provide easier portability.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112, sixth paragraph, unless and until such claim limitations expressly use the phrase “means for” followed by a statement of function void of further structure.

What is claimed is:

1. An ultrasound probe holder for use with a portable ultrasound system, comprising:
a probe holder being configured to have first and second interconnection portions, the first interconnection portion being configured to interconnect with a three-dimensional (3D) object; and
a second opening being formed in the second interconnection portion, the second opening being configured to accept a scanning end of an ultrasound probe.

2. The probe holder of claim 1, wherein the first interconnection portion further comprises a first opening having an inner height and an outer height, the inner height being greater than the outer height, the first opening being configured to receive a connector end of the ultrasound probe.

3. The probe holder of claim 1, wherein the first interconnection portion further comprises a first opening having an inner portion, the first opening having top and bottom portions extending substantially parallel to each other along the inner portion, the top and bottom portions curving at an outer edge beyond the inner portion to form a C-shape.

4. The probe holder of claim 1, wherein the 3D object is one of a connector end of the ultrasound probe, a table top, a case for carrying the ultrasound probe, a case for carrying the portable ultrasound system, and an edge of the portable ultrasound system.

5. The probe holder of claim 1, wherein the first interconnection portion further comprises a first opening being configured to slidingly receive a connector end of the ultrasound probe.

6. The probe holder of claim 1, wherein the probe holder further comprises a top and a bottom that are opposite to each other and first and second sides that are opposite to each other, wherein the second opening is configured to accept the scanning end of the ultrasound probe from an angular direction proximate to the top, the angular direction being approximately 45 degrees with respect to a plane extending parallel to the bottom of the probe holder.

7. The probe holder of claim 1, wherein the second opening having a back wall and top and bottom inner surfaces, the back wall forming a first angle with the top inner surface and a second angle with the bottom inner surface, wherein the first angle is less than approximately 90 degrees and the second angle is approximately 90 degrees.

8. The probe holder of claim 1, wherein the probe holder is one of a unitary construction and an assembly comprising more than one piece of material joined together.

9. A portable ultrasound system comprising:
a portable ultrasound system for acquiring ultrasonic data of a patient;
an ultrasound probe connectable to the ultrasound system for scanning the patient, the ultrasound probe having a connector end for interfacing with the system and a scanning end for scanning the patient, the connector end and the scanning end being interconnected with a cable; and
a probe holder for holding at least the scanning end of the ultrasound probe when the scanning end is not being used to scan the patient.

10. The system of claim 9, wherein the probe holder further comprises first and second openings for interfacing with a 3D object and the scanning end of the probe, respectively, the probe holder being releasably attachable with respect to the 3D object and the scanning end.

11. The system of claim 9, wherein the probe holder further comprises an interconnecting portion, the portable ultrasound system further comprising an opening for releasably accepting the interconnecting portion.

12. The system of claim 9, wherein the probe holder further comprises an opening configured to be releasably attachable to a three-dimensional object.

13. The system of claim 9, wherein the probe holder further comprises an opening configured to be releasably attachable to at least one of the connector end of the ultrasound probe, a case for carrying the ultrasound probe, a case for carrying the portable ultrasound system, and a table top.

14. The system of claim 9, wherein the probe holder is formed of a material having flexible properties, the probe holder having an opening configured to be releasably attachable to the connector end of the ultrasound probe, the opening flexing to accept and release the connector end.

15. An ultrasound probe holder, comprising:
a central region having a top and a bottom that are opposite to each other and first and second sides that are opposite to each other; and
top and bottom portions extending from the central region in both first and second directions, the top and bottom portions forming first and second openings in the first and second sides, respectively, the first and second openings configured to releasably hold a connector end and a scanning end, respectively, of an ultrasound probe.

16. The probe holder of claim 15, wherein the central region further comprises front and back sides, wherein the first and second openings are open to the front and back sides.

17. The probe holder of claim 15, wherein the bottom is configured to rest on a surface.

18. The probe holder of claim 15, wherein the second opening is configured to open in an angular direction between the top and the second side and the first opening is configured to open towards the first side.

19. The probe holder of claim 15, wherein the top and bottom portions forming the second opening flex outward with respect to each other when the scanning end is inserted there-between.

20. The probe holder of claim 15, wherein the top and bottom portions forming the first opening are configured to slide over the connector end.
